(19) United States
(12) Patent Application Publication
(10) Pub. No.: US 2023/0172552 A1
(43) Pub. Date: Jun. 8, 2023

(54) CARDIOVASCULAR MONITORING SYSTEM

(71) Applicants: Thomas Jefferson University, Philadelphia, PA (US); RTM Vital Signs, LLC, Philadelphia, PA (US)

(72) Inventors: Jeffrey I Joseph, Penn Valley, PA (US); Josiah Verkaik, Boise, ID (US); Denise Devine, Media, PA (US)

(21) Appl. No.: 18/071,860

(22) Filed: Nov. 30, 2022

Related U.S. Application Data
(60) Provisional application No. 63/285,149, filed on Dec. 2, 2021.

(51) Int. Cl. A61B 5/00; A61B 5/0215; A61B 5/021

(52) U.S. Cl. CPC A61B 5/6876; A61B 5/6879; A61B 5/6885; A61B 5/0215; A61B 5/02141; A61B 2560/063

(57) ABSTRACT

A method of associating a sensor with a blood vessel includes providing a sensor defining a passage therethrough and at least partially encircling the outside wall of the blood vessel with at least a portion of the sensor. The method further includes compressing the passage through the sensor until a portion of the blood vessel is flattened and mechanically coupling a pressure/force transducer to the outside wall of the blood vessel in an area of vessel wall flattening.

### CROSS-REFERENCE TO RELATED APPLICATION

This application claims the benefit of U.S. Application Ser. No. 63/285,149, filed Dec. 2, 2021.

### FIELD

This disclosure relates to a method and system for an implantable cardiovascular monitoring system.

### BACKGROUND

More than 1,000 people die suddenly every day in the United States. In August 2020, the National Center for Biotechnology Information (NCBI) estimated that up to 80% of sudden deaths outside of the hospital were related to cardiovascular disease. Essentially, the vital signs of none of these victims were monitored in real-time or recorded for later analysis.

Recent advances in microelectromechanical systems (MEMS) sensors, electronics, flash memory, rechargeable battery power, telemetry, software, and data analytics have led to an increased interest in the development and commercialization of a long-term implantable cardiovascular monitoring system with clinical accuracy, particularly for those at highest risk for an adverse event due to uncontrolled hypertension and cardiovascular disease.

Cardiovascular disease is the leading cause of morbidity and mortality world-wide. Hypertension, also known as high blood pressure (BP), is the number-one controllable risk factor for fatal strokes, fatal heart attacks, congestive heart failure, chronic kidney failure, dementia, and sudden death. Nearly half of all adults in the United States have hypertension, while only about 25% have their disease under control. Approximately 13 million people in the United States have resistant hypertension, defined as uncontrolled hypertension despite treatment with diet, exercise, and three or more anti-hypertensive medications. Despite better medications for treating hypertension, poorly controlled blood pressure remains the major cause of premature death in adults—making it responsible for every 1 in 8 deaths. More timely and effective diagnosis and treatment of chronic cardiovascular disease, especially uncontrolled hypertension, causes a significant decrease in the incidence and severity of serious adverse cardiovascular events.

Arteriolar sclerosis and atherosclerosis are often cited by researchers as the mechanism by which chronic hypertension causes artery and arteriole stiffening, and chronic/acute arterial blood flow obstruction (myocardial infarction, stroke, peripheral vascular disease). In addition, the increased peripheral vascular resistance caused by hypertension leads to left ventricular hypertrophy, ischemic heart disease, systolic/diastolic heart failure, and aortic/mitral valve insufficiency.

The measurement of BP using an upper arm blood pressure cuff, is routinely done in a doctor's office or clinic using manual or oscillometric techniques. Infrequent clinic measurements of the systolic, mean, and diastolic blood pressure at rest remain the most common method for diagnosing and managing patients with hypertension. Automated oscillometric devices are now available that use an upper arm cuff to measure the systolic, mean, and diastolic BP of ambulatory patients every 30 to 60 minutes for 24 to 48 hours. Ambulatory BP monitoring has gained clinical acceptance to diagnose and manage hypertension, especially in patients that may have “white coat” hypertension that resolves once outside of the physician's office. The use of ambulatory BP monitoring is time-limited due to the discomfort of cuff inflation and the inconvenience of wearing a bulky device. However, an ambulatory patient's arterial BP fluctuates constantly in response to the physiological stimuli of everyday living, and as such, the intermittent measurement of the arterial BP at rest as the most important risk factor for cardiovascular disease appears inconsistent with a large body of data on hypertension. In addition, the focus on an elevated mean BP measured is questionable, because an elevated systolic pressure, a variable BP, and an increased pulse pressure have all been associated with an increased risk for a cardiovascular adverse event.

Recently, there has been an increased interest in the importance of BP variability rather than mean BP in predicting the risk for cardiovascular disease. Re-analysis of large clinical trials has shown that the variability between in-office BP measurements is a predictor of cardiovascular events, independent of the mean BP. The measurement devices used in these trials provided few data points at rest, and were unable to measure the dynamic and transient changes in BP that occur during daily activities.

Physicians frequently recommend that patients self-monitor their BP at home using an oscillometric device with an upper arm cuff, or an inflatable cuff around the wrist. Unfortunately, an accurate measurement requires the patient to remain still with the device held at the heart level for more than 60 seconds. Modern devices can communicate wirelessly with a central monitoring station and electronic medical record with computer data analytics to enhance the clinical efficacy of remote patient monitoring.

Existing non-invasive wearable devices utilize pulse transit time to estimate arterial blood pressure. The devices measure the electrocardiogram to detect the onset of systole and measure the onset of the peripheral pulse wave using photo-plethysmography. The measured pulse transit time correlates with the arterial blood pressure, but is also influenced by arterial elasticity or compliance. The technology of pulse transit time is flawed because the slope of the relationship with arterial BP differs between individuals and in the same individual during physiological perturbations and is very sensitive to motion artifacts. Additionally, known wearable monitors using pulse transit time require patients to remain still for 60 seconds during a measurement, frequent calibration with an upper arm cuff, and are not accurate enough to diagnose hypertension, or dose medication.

Physicians in the hospital routinely insert a vascular catheter into the lumen of a radial, brachial, or femoral artery to continuously monitor the arterial blood pressure waveform. The catheter is attached to fluid filled tubing and an external pressure transducer hard-wire connected to a bedside data acquisition system and display. Invasive arterial blood pressure monitoring has been performed in the emergency rooms, operating rooms, and intensive care units of hospitals for more than 50 years to enhance the timeliness and accuracy of diagnosis, with the goal of preventing a serious adverse event. Recent advances in arterial waveform analysis provide diagnostic information for calculating important heart function parameters beyond systolic, mean, and diastolic BP, and is more accurate than intermittent BP cuff measurements. In addition, the arterial BP waveform can be analyzed to determine a patient's heart rate, stroke volume, cardiac output, myocardial contractility, systemic vascular resistance, systolic/diastolic timing intervals, and valve function—important metrics for diagnosing and treating chronic cardiovascular disease. A few clinical trials in ambulatory outpatients with hypertension have used a brachial arterial catheter, external pressure transducer, and a data logger to record the dynamic changes in the arterial blood pressure waveform over a 24 to 48 hour period. The risk of arterial bleeding and infection limit this method to short-term monitoring of highly compliant patients.

Arterial BP is routinely measured for clinical care using an upper arm cuff device with the patient sitting still with their feet on the floor for at least 5 minutes. In contrast, dynamic patterns have been identified during ambulatory BP monitoring in healthy and hypertensive patients: 1) normal 20% to 30% decrease in BP during sleep, 2) normal 20% to 30% increase in BP upon awakening, 3) normal >20% increase in BP during daily activities, 4)>30% increase in BP during exercise, and 5) a lack of BP decrease during sleep and a persistent increase in BP during daily activities in patients with uncontrolled hypertension. In addition, many patients experience a significant decrease on BP with symptoms due to medications, dehydration, and upon moving from the supine to the standing position (orthostatic hypotension). Even a brief period of hypotension can cause end-organ damage of the brain, heart, and kidneys.

### SUMMARY

The techniques of this disclosure generally relate a method, device, and system for cardiovascular monitoring.

In one aspect, a method of associating a sensor with a blood vessel, comprising providing a sensor defining a passage therethrough; at least partially encircling the outside wall of the blood vessel with at least a portion of the sensor; and compressing the passage through the sensor until a portion of the blood vessel is flattened, and mechanically coupling a pressure/force transducer to the outside wall of the blood vessel in an area of vessel wall flattening.

In another aspect, the sensor comprises a first portion and a second portion, each defining a portion of the passage through the sensor for cooperatively encircling the blood vessel, providing a surgical implantation tool that positions the first portion and the second portion of the sensor on opposite sides of the blood vessel in a selected x-y-z alignment, and positioning the sensor within the area of vessel wall flattening.

In another aspect, the method further comprises urging the first portion toward the second portion to join the first portion and the second portion, thereby encircling the blood vessel, and flattening a portion of the blood vessel.

In another aspect, flattening a portion of the blood vessel causes at least 10% applanation of the portion of the blood vessel.

In another aspect, flattening a portion of the blood vessel causes at least 15% application of the portion of the blood vessel.

In another aspect, flattening a portion of the blood vessel causes at least 25% application of the portion of the blood vessel.

In another aspect, the method further comprises promotion of tissue ingrowth into at least a portion of the sensor to enhance mechanical coupling of the sensor to the blood vessel in the area of flattening.

In another aspect, the passage therethrough of the sensor encircles the blood vessel with at least a portion of the sensor and reduces the diameter of the passage through of the sensor until a portion of the blood vessel is flattened and the pressure/force transducer measures pressure within the blood vessel and outputs a signal to an electronic module that analyzes and displays the blood vessel blood pressure waveform and calculates blood pressure based upon a sensed movement.

In one aspect, a method of surgically implanting a sensor within a human body, comprises locating a peripheral artery at the approximate level of an aortic valve, measuring an outer diameter of the peripheral artery with ultrasound, providing a support structure having a tissue ingrowth surface for the peripheral artery, positioning a portion of the peripheral artery within the support structure, positioning a sensor module over the portion of the peripheral artery with sufficient force to deform the peripheral artery and to join the sensor module to the support structure, and joining the support structure to the sensor module to maintain the deformation of the peripheral artery.

In another aspect, the method further comprises promoting tissue ingrowth of the portion of the peripheral artery sufficient to couple the portion of the peripheral artery to one of the support structure and the sensor.

In another aspect, the force to deform the portion of the peripheral artery is sufficient to cause at least 10% applanation of the portion of the peripheral artery.

In another aspect, the force to deform the portion of the peripheral artery is sufficient to cause at least 15% applanation of the portion of the peripheral artery.

In another aspect, the force to deform the portion of the peripheral artery is sufficient to cause at up to 25% applanation of the portion of the peripheral artery.

In one aspect, a method of surgically implanting a sensor within a human body, comprises locating a peripheral artery at the approximate level of an aortic valve; measuring an outer diameter of the peripheral artery with ultrasound; choosing an optimum size of a sensor module and a support structure; providing the support structure having a tissue ingrowth surface for the peripheral artery; positioning a portion of the peripheral artery within the support structure; positioning the sensor module over the portion of the peripheral artery with sufficient force to deform the peripheral artery and to join the sensor module to the support structure; and joining the support structure to the sensor module to maintain the deformation of the peripheral artery; and mechanically securing an array of force transducers to the outer surface of the blood vessel wall.

In another aspect, the sensor module is positioned over the portion of the peripheral artery with sufficient force to deform the peripheral artery and the sensor module is joined to the support structure, the sensor module and the support structure cooperatively encircle the blood vessel.

### BRIEF DESCRIPTION OF THE DRAWINGS

A more complete understanding of embodiments described herein, and the attendant advantages and features thereof, will be more readily understood by reference to the following detailed description when considered in conjunction with the accompanying drawings wherein:

FIG. 1 is a perspective view of an embodiment of an implantable cardiovascular monitoring system constructed in accordance with the principles of the present application and a view of a sensor module and sensor module retaining device coupled to a patient's blood vessel. The sensor module is connected to an implantable electronics module (not shosawn) via a flexible lead. The electronics module communicates wirelessly with an external smart device (phone, tablet, personal computer, or upper arm cuff BP monitor). The external upper arm cuff BP monitor can be used to intermittently calibrate the implanted force/pressure sensor;

FIG. 2 is a perspective view showing the sensor module coupled to sensor module retaining device around a peripheral blood vessel (artery);

FIG. 3 is a cross-sectional view showing how the sensor module retaining device of FIG. 1 is sized and configured to contour around an outer surface of the patient's blood vessel, and how the coupling of the sensor module retaining device and the sensor module results in applanation of the blood vessel with the diaphragm of the force/pressure transducer located within the area of vessel wall flattening;

FIG. 4 is a perspective view of the sensor module retaining device of FIGS. 1-3;

FIG. 5 is a cross-sectional view showing the coupling of the sensor module and the sensor module retaining device around the outside wall of a blood vessel, aligning the diaphragm of the force/pressure transducer within the area of flattening;

FIG. 6 is a flow chart showing steps in a method of using the exemplary embodiments;

FIG. 7 illustrates a surgical tool useful for implanting the exemplary embodiments into a human body;

FIG. 8 illustrates an embodiment of the sensor module with >10% applanation;

FIG. 9 illustrates another embodiment of the sensor module with <10% applanation;

FIG. 10 illustrates another embodiment of the sensor module with >10% applanation;

FIG. 11 illustrates another embodiment of the sensor module without applanation;

FIG. 12 illustrates an inner surface of a sensor module with an array of miniature force transducers (rather than the diaphragm of one force/pressure transducer); and

FIG. 13 illustrates the cross-section view of the sensor module of FIG. 12 showing the array of miniature force transducers coupled to the outer surface of the blood vessel in the area of applanation.

### DETAILED DESCRIPTION

Before describing in detail exemplary embodiments, it is noted that the embodiments reside primarily in combinations of apparatus components and processing steps related to an implantable cardiovascular monitoring system. Accordingly, the system and method components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

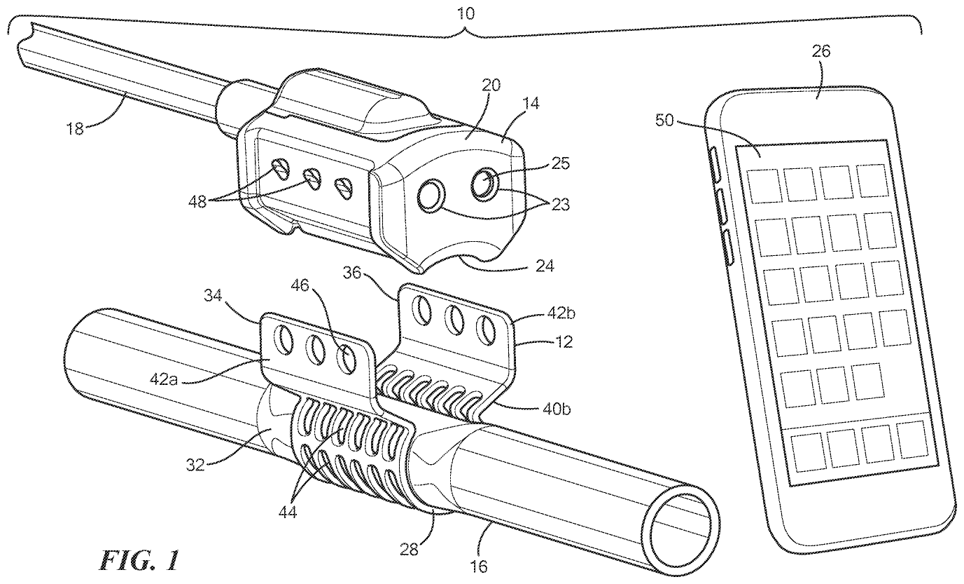

Referring now to FIGS. 1-5, is an implantable Cardiovascular Monitoring System (CMS) 10 for measuring the vital signs of a patient having a blood vessel is shown. As shown in FIG. 1, the system 10 includes a sensor module retaining device and/or the support structure 12, sized and configured to receive, retain, and removably couple a sensor module 14 to an artery or target blood vessel 16 within the body of a patient. Upon implantation, the sensor module retaining device 12 may surround select portions of the blood vessel 16 without necessitating the complete encircling of the blood vessel 16. The sensor module 14 may be an arterial applanation tonometer fully or partially implanted within the patient and having at least one sensor (as described in more detail below) configured to continuously measure a blood pressure waveform from the target blood vessel 16, and is connected to an implantable electronic module or electronics communication assembly (not shown) via a flexible electric lead 18. However, in other configurations, in addition to continuously measuring a patient's blood pressure waveform, the sensor module 14 may also be configured to continuously measure and analyze the patient's electrocardiogram, hemoglobin oxygen saturation, photo-plethysmograph waveform (pulse oximeter), temperature, respiratory rate, tidal volume, breathing pattern, body position, activity level, and sounds of the upper airway, lungs, and heart (phonocardiogram). Although not shown, it us to be understood that the electronics module can generally contain a motherboard with integrated circuit (IC) chips, control software, diagnostic software, memory, a transmitter/receiver, an antennae, and a rechargeable battery.

The sensor module 14 may be made from a biocompatible, non-degradable, material such as titanium, stainless steel, MP35N alloy, plastic, or ceramic having a low density, relatively high strength, and a relatively high level of corrosion resistance. When coupled together, the sensor module 14 and sensor module retaining device 12 form a fixed (non-compliant) interface or structure. The sensor module 14 may be implanted around the outer surface of any peripheral artery or target blood vessel (i.e., peripheral artery, internal thoracic/mammary artery, superior thoracic artery, thoracoacromial artery, thoracic-dorsal artery, lateral thoracic artery, external carotid artery, and the superior epigastric artery, etc.) using manual surgical methods and/or a customized surgical tool configured for positioning and attachment of the sensor module to the target blood vessel in the optimal x-y-z alignment and degree of applanation/flattening). An electronics module (not shown) may then be implanted within the subcutaneous tissue of the chest wall of the patient. The sensor module 14 may also be implanted using local anesthesia with sedation and aseptic surgical technique. The clinician may use external ultrasound to image the outer diameter of the target artery (i.e., the target blood vessel 16) prior to surgical implantation. Based upon the ultrasound measurements, the surgeon may choose a width of the sensor module 14 and an inner diameter of the sensor module retaining device 12 to produce an amount of applanation (10% to 25% flattening) of the target blood vessel 16. Thus, the sensor may be manufactured with a range of widths and the sensor module retaining device may be manufactured with a range of internal diameters. The sensor module 14 can be implanted around the outside of the target blood vessel 16 manually or using a J-shaped surgical tool 180. Implanting the sensor module 14 approximately at the level of the aortic valve will minimize the effect of a change in body position (gravity) on the arterial BP waveform measurement.

Although not described in detail herein for simplicity, it is to be understood that the sensor module 14 generally includes a housing 20 sized and configured to retain at least one pressure/force sensor 21, temperature thermistor, silicone fluid reservoir, and processing circuitry (not shown), and a diaphragm 22 coupled to the inferior surface 24 of the housing 20. In one exemplary configuration, for example, as shown in FIG. 1, the housing 20 may have a width larger than a width or diameter of the target blood vessel 16. However, in other configurations, the housing 20 may also have a width equal to or smaller than the width or diameter of the target blood vessel 16.

Surgical implantation of the arterial applanation tonometer sensor head and sensor module retaining device 12 can be accomplished using a manual method or a surgical tool. The coupling of the sensor module's 14 diaphragm 22 to the outside wall of the target blood vessel 16 in a region of applanation or flattening can impact long-term performance. The sensor module retaining device 12 must maintain mechanical coupling of the diaphragm 22 within the flattened region of target blood vessel 16 over the physiological range of vasodilation and vasoconstriction. For example, a 2.5 mm outer diameter peripheral artery may increase 700 micrometers during systole and decrease 700 um in diameter during diastole. The target blood vessel 16 can increase up to 20% in outer diameter due to a moderate increase in systolic/diastolic/mean blood pressure, and decrease up to 50% in outer diameter due to vasoconstriction (spasm) or very low blood pressure. A significant decrease in outer diameter may adversely affect mechanical coupling of the diaphragm 22 to the outer wall of the target blood vessel 16.

The sensor module retaining device 12 defines openings or apertures (discussed below) that faciliate the ingrowth of adjacent connective tissue and artery wall tissue that can help secure/couple the sensor module retaining device 12 with the vessel wall tissue. In addition, the inferior surface 24 of the sensor module 14 can also have a textured or porous architecture designed to facilitate further ingrowth of adjacent arterial wall tissue. Tissue that grows through the openings or apertures of the sensor module retaining device 12, and grows into the sensor module's 14 textured inferior surface 24, mechanically secures the diaphragm 22 to the wall tissue of the target blood vessel 16. This particular configuration produces a strong and robust coupling of the diaphragm 22 to the wall of the target blood vessel 16 despite vasocontriction and eliminates body motion induced artifact or noise. The ingrowth of adjacent connective tissue also enhances the mechanical strength of the wall tissue of the target blood vessel and blood supply of the target blood vessel (vaso vasorum).

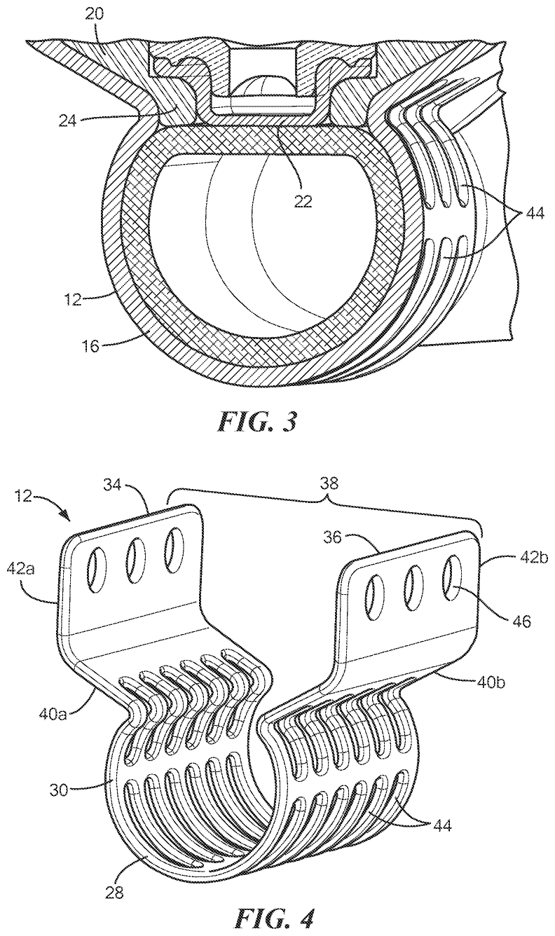

As shown in FIG. 3, when coupled to the sensor module retaining device 12, the sensor module 14 is positioned such that the diaphragm 22 is in contact with the outer surface of the blood vessel 16, which allows the pressure sensor 21 disposed within the sensor module 14 to measure a change in the intravascular hydraulic pressure when a force is imparted on the diaphragm 22 by the blood vessel wall 16. In other words, pressure sensor 21 is configured to measure at least one blood pressure waveform from the target blood vessel 16. Once the blood pressure waveform of the blood vessel 16 is measured and recorded, the processing circuitry of the sensor module transmits the recorded measurements to a remote-control unit 26 (discussed in more detail below) via a low-energy Bluetooth® signal or other wireless protocol such as the Medical Device Radiocommunication Service (MICS).

Although not shown in detail herein, the housing 20 may define a non-compliant fluid chamber sealed by the diaphragm 22. A fluid, such as silicone fluid, may be disposed within the fluid chamber for exhibiting a hydraulic pressure consistent with the pressure applied to the diaphragm 22. Hydraulic generally refers to fluid in a confined space (closed system) wherein the fluid is a medium to transmit force. This is in accordance with the discovery of Pascal that a pressure applied to any part of a confided fluid transmits to every other part with no loss. The pressure acts with equal force on all equal areas of the confining walls in a direction perpendicular to the wall surfaces. The pressure sensor 21 of the sensor module 14 located on the outside wall of the blood vessel is configured to measure a change in the hydraulic pressure when the force is imparted on the diaphragm 22, such as on the inferior surface 24, or any other outer surface, of the diaphragm 22 by the blood vessel wall 16. As such, the diaphragm 22 may be a compliant diaphragm configured to be secured, i.e., mechanically coupled, against a wall of the blood vessel 16 to facilitate transduction of the blood pressure through a wall of the blood vessel 16. The pulsatile intravascular blood pressure is transmitted across the flattened artery wall tissue and across the diaphragm 22 into the silicone fluid filled reservoir without distortion or loss of energy. The diaphragm is sandwiched between the flattened vessel wall tissue and the fluid within the non-complaint chamber. The pressure is transferred across the diaphragm into the non-complaint fluid without macroscopic movement of the diaphragm material. The MEMS pressure die measures the pulsatile pressure waveform within the reservoir with satisfactory accuracy for detecting and diagnosing worsening cardiovascular disease (hypertension, heart failure, myocardial ischemia, valve disease, pulmonary embolism, etc.) and dosing medication.

In some configurations, the hydraulic pressure within the diaphragm 22 can be measured by the pressure/force sensor 21 of the sensor module 14, with the pressure sensor 21 being a resistive or capacitive sensor, for example, a MEMS sensor or MEMS pressure die. As a non-limiting example, the pressure sensor 21 may be that which is marketed and sold under the name NovaSensor® or the pressure sensor 21 may be a silicon, micro-machined, piezo resistive pressure sensing chip within the Smi510E Series, such as, for example SM5108E, owned by Silicon Microstructures, Inc. In other configurations, the pressure sensor 21 may be another type of sensor configured to measure hydraulic pressure. In an alternate configuration, the inner surface of the sensor module 14 can be made from a single piezoelectric force transducer or a miniature array of force transducers (for example 4, 16, 36, 72, 144 force transducers on a 1 mm×6 mm computer chip) coupled to the outer artery wall of the target blood vessel 16 within a region of applanation (see drawing below).

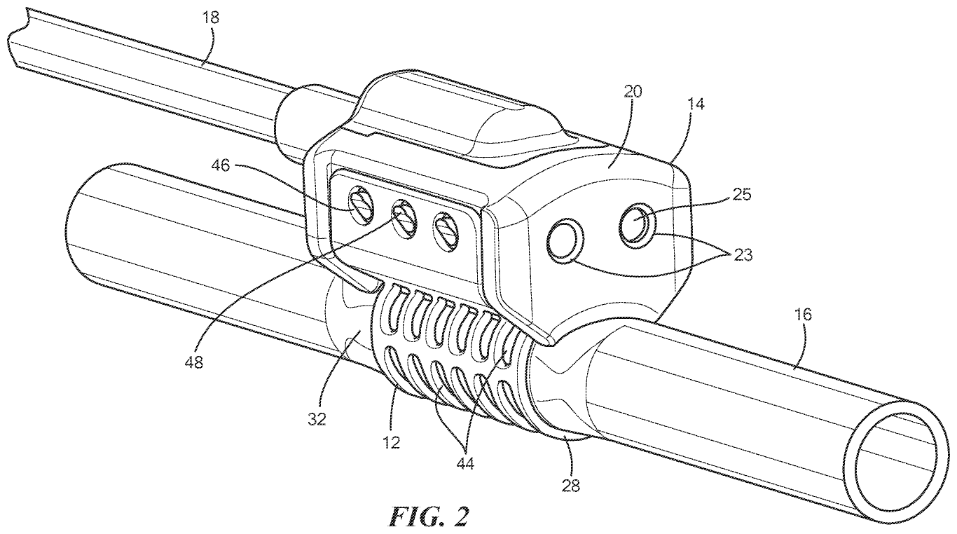

As shown in FIGS. 1 and 2, in order to insert the fluid within the fluid chamber, the housing 20 defines one or more ports 23, for example a fill port and a vent port, in fluid communication with the fluid chamber. The fill port may be used to deposit and adjust the amount of the fluid, e.g., silicone, located within the fluid chamber, whereas the vent portion may be used to receive a vacuum to remove any entrapped air from the fluid chamber. The removal of air assists in providing the fluid that is free of air bubbles in an effort to obtain accurate hydraulic pressure measurements. As shown in FIGS. 1 and 2, a stopper 25, for example a ceramic ball, may be disposed within each of the ports 23 to provide a hermetic seal.

Referring now to FIG. 4, in some configurations, the sensor module retaining device 12 may be sized and configured to minimize the change in lumen shape and cross-section area of the target blood vessel 16. The sensor module 14 may be connected to and disconnected from the superior arms (e.g., the second and third portions 34, 36, discussed below) of the sensor module retaining device 12. The inner diameter of the sensor module retaining device 12 may impact the amount or degree of applanation. The artery wall tissue of the target blood vessel 16 will remodel to take on the D-shape of the lumen produced by the connected sensor module 14 and sensor module retaining device 12. The sensor module 14 can be manufactured with one size (for example 8 mm length×1.6 mm width×8 mm height), while the sensor module retaining device 12 can be made with a wide range of inner diameter sizes to accommodate an artery with an outer diameter 1.8 mm to 3.2 mm (sensor module retaining devices manufactured with a 200 μm larger or smaller diameter). The sensor module 14 can be manufactured with a range of widths (1.2 mm, 1.4 mm, and 1.6 mm) to accommodate a range of artery outer diameters. The sensor module 14 with a smaller width could be used for a smaller artery and a larger width could be used for a larger artery.

Continuing to refer to FIG. 4, an exemplary sensor module retaining device 12 is shown that is made out of a biocompatible, non-degradable, material such as titanium, stainless steel, MP35N alloy, plastic, or ceramic with, relatively high strength, and corrosion resistance. The sensor module retaining device 12 includes a first portion 28 that defines an open loop 30 sized and configured to contour around an outer surface 32 of the target blood vessel 16. In other words, the open loop 30 is defined by a curvature of the first portion 28 such that at least a portion of the first portion 28 may be flush with and curve along the outer surface 32 of the target blood vessel 16. In one embodiment, the open loop 30 defined by the first portion 28 may have a diameter sufficiently large to receive and couple to a target blood vessel having an outer diameter between at least 1.8 mm to 3.2 mm. The sensor module retaining device 12 also includes a second portion 34 and a third portion 36 opposite the second portion 34. As shown in FIG. 4, the second and third portions 34, 36, are each contiguous with and converge or taper inwards towards the first portion 28. The open loop 30 is defined by the first portion 28 between second and third portions 34, 36. Also, the second and third portions 34, 36, each extend distally away from the open loop 30 and are sized and configured to define an open channel 38 therebetween. As shown in FIGS. 1, 4, and 5, the defined open channel 38 is sized to receive the sensor module 14. In embodiments wherein the sensor module 14 has a width larger than the width of the target blood vessel 16, the open channel 38 may also have a width larger than the width of the target blood vessel 16. As such, the open channel 38 may therefore also have a width larger than a width of the open loop 30. Further, as described herein, it is to be understood that the first, second, and third portions 28, 34, 36 of the sensor module retaining device 12 may each have rounded or curved edges to minimize the potential for sharp edges to be in contact with soft tissue of a target blood vessel or other surrounding tissue structures.

Continuing to refer to FIG. 4, the second and third portions 34, 36, may each include multiple contiguous segments. For example, the second and third portions 34, 36, may each include a proximal segment 40a, 40b, and a distal segment 42a, 42b, respectively. The distal segments 42a, 42b, are substantially orthogonal or angled in relation to each respective proximal segment 40a, 40b. The proximal segment 40a, 40b of each portion 34, 36, is proximate to the first portion 28. The distal segments 42a, 42b of each portion 34, 36, are distal to and extend distally away from the first portion 28. The proximal segments 40a, 40b each converge or taper inwards from the distal segments 42a, 42b towards the first portion 28.

In some configurations, the sensor module retaining device 12 may be substantially U-shaped such that it may contour around the perimeter (outside wall) of the target blood vessel 16. The sensor module retaining device 12 and the sensor module 14 can be pre-loaded on a substantially J-shaped surgical tool 180. When implanting the device 12, the clinician can isolate a 10 mm length of the target blood vessel 16 and remove the outer tunica adventitia using routine surgical methods. The target blood vessel 16 can be advanced through the gap located between the inferior surface 24 of the sensor module 14 and the sensor module retaining device 12. The target blood vessel 16 becomes located within the sensor module retaining device 12. The tool can be “fired” to connect the sides of the sensor module 14 to the second and third portions 34, 36 of the sensor module retaining device 12 in the optimal x-y-z alignment and degree of applanation. The implantation tool can then be removed from the surgical site and the sensor head's lead attached to an implantable electronics module.

Now referring to FIGS. 1, 4, and 5, the entirety of the sensor module retaining device 12, or at least the first portion 28, may be textured, surface modified, surface coated with a biocompatible material, or scaffold-like to facilitate the adhesion and ingrowth of wall tissue of the target blood vessel 16 and adjacent connective tissue. Also, the first portion 28 may define a plurality of openings or apertures 44. The plurality of apertures 44 may be arranged uniformly (as shown in FIG. 1) or in any other arrangement or pattern along the curvature of the first portion 28, and facilitate the ingrowth of adjacent tissue and vaso vasorum (blood supply) to the wall tissue of a target blood vessel 16. For example, when the sensor module retaining device 12 is coupled to the outside wall of the target blood vessel 16, cells and connective tissue from the target blood vessel 16 and adjacent tissue may continue to grow in and between each aperture 44, thereby further binding the sensor module retaining device 12 to the target blood vessel 16. Thus, tissue integration will enhance robust mechanical coupling of the artery wall tissue to the pressure/force sensor's diaphragm despite a decrease in vessel diameter (due to intravascular hypotension or vasoconstriction), and eliminate body motion induced artifact (noise). The vascular tissue ingrowth will also enhance the strength, thickness, and health of the vessel wall tissue. Similarly, the inferior surface 24 of the housing 20 may be textured, surface modified, or scaffold-like to facilitate the adhesion and ingrowth of wall tissue of a target blood vessel.

Additionally, in one embodiment, at least a portion of the sensor module retaining device 12 or sensor module 14 may be coated with chemicals, medications, or other types of therapeutic agents or drugs that promote the ingrowth and adhesion of wall tissue and/or minimize acute and chronic inflammation. The adhesion or ingrowth of the wall tissue of a target blood vessel to the surface of the sensor module 14 enhances the mechanical coupling of the sensor module's 14 diaphragm 22 to the outer wall or surface 32 of the blood vessel 16—especially when the blood vessel diameter becomes smaller due to vasoconstriction or low blood pressure. In other words, the desired effect of the tissue adhesion or ingrowth is reliable mechanical coupling of the sensor module diaphragm 22 to a target blood vessel, while producing the smallest amount of applanation.

As shown in FIGS. 1, 4, and 5, the distal segments 42a, 42b of the second and third portion 34, 36, may further define at least one opening 46 sized to engage or receive at least one corresponding mating member 48 on the housing 20 of the sensor module 14. Each corresponding mating member 48 may be a button, stem, rod, or other type of protrusion sized and configured to be received through an opening 46. In one embodiment, the sensor module 14 is coupled to the sensor module retaining device 12 via a snap-fit connection between the openings 46 and the corresponding mating members 48 on the housing 20.

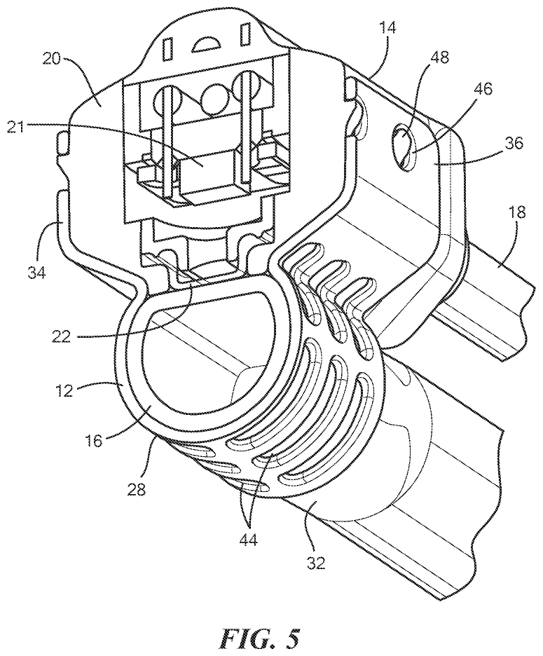

Now referring to FIG. 5, the sensor module 14 is received within the open channel 38 and coupled to the sensor module retaining device 12 via the mating of the openings 46 and the at least one corresponding mating members 48. When the sensor module 14 is coupled to the sensor module retaining device 12, the sensor module 14 is adapted to minimize stress imposed on the blood vessel 16 in undergoing a forced transition from a circular geometry to a constrained flattening of the blood vessel (as shown in FIGS. 3 and 5) for achieving applanation and accurate blood pressure transduction through the blood vessel wall to the diaphragm 22. The design of the second and third portions 34, 36 of the sensor module retaining device 12 enable the sensor module 14 to least partially surround the blood vessel 16 without overly constraining the blood vessel 16 when obtaining the blood pressure measurements or performing alternative medical diagnostics. When fully coupled to the sensor module retaining device 12, the inferior surface 24 of the sensor module 14 is in contact with the outer surface 32 of the target blood vessel 16 to produce 10% to 15% to 25% applanation of the blood vessel 16.

In one embodiment the inferior surface 24 of the sensor module 14 may have fixed width of approximately 1.6 mm (diaphragm 22 has 1 mm width and the housing 20 has a 0.3 mm width on each side). The pulsatile intra-vascular pressure is transmitted across the wall tissue of the blood vessel 16 and diaphragm 22 into the silicone fluid reservoir with minimal distortion or attenuation. The inferior surface 24 of the sensor module 14 and sensor module retaining device 12 may be textured to facilitate adhesion of the blood vessel tissue to the sensor module 14. Although not shown, the inferior surface 24 of the sensor module 14 may also include a light source and detector of an implantable pulse oximeter to continuously measure the hemoglobin oxygen saturation carried in the patient's red blood cells and a photo-plethysmograph waveform.

Continuing to refer to FIGS. 1 and 5, when fully coupled to the sensor module retaining device 12 and in contact with the target blood vessel 16, the sensor module 14 may continuously record the blood pressure waveform of the blood vessel 16. The measurements are then transmitted to an external control unit 26 via a low-energy Bluetooth® signal or a similar method of electronic data transmission. In some configurations, the control unit 26 can be connected to an external oscillometric BP monitor with an upper arm cuff. In some such configurations, the patient or clinician can intermittently calibrate the arterial BP waveform (systolic, mean, and diastolic BP) of the implanted sensor module 14 using one or more measurements made by the sensor module 14. The calibration data can be transmitted to the sensor module 14 as frequently as necessary (for example every 7 to 28 days) to produce a BP measurement accurate enough to diagnose hypertension, hypotension, and dose medication. In other configurations, the control unit 26 may be an external control unit operated by computer or by the clinician and/or the patient. In some configurations, the control unit 26 may also be a smart phone, smart watch, tablet, computer, or other type of smart device. Further, it is to be understood that in one embodiment, the sensor module 14 may be in communication with more than one remote control unit 26. For example, the sensor module 14 may be in communication with a patient's smart phone and an external control unit operated by a clinician. Once the blood pressure waveform signals are transmitted to the remote control unit 26, the processing circuitry of the remote control unit 26 may then analyze and display in real-time the recorded blood pressure waveform measurements and also display any visual and/or audible alerts and alarms on a display or screen 50 of the remote control unit 26. The alerts and/or alarms may be indicative of an adverse event such as low, high, and/or abnormal blood pressure readings or patterns that are being observed or have been observed by the sensor module 14. As such, the alerts and/or alarms may also notify clinicians and/or patients as to whether the patient's levels of certain blood pressure regulating medications that need to be adjusted, such as to treat low, high, or abnormal blood pressure, or whether the patient needs to seek medical attention.

In a broader sense, the implantable Cardiovascular Monitoring System (CMS) can simultaneously measure, analyze, and display trend data from multiple vital sign sensors (electrocardiogram, pulse oximeter, temperature thermistor, microphones, accelerometer) that continuously monitor an ambulatory patient's cardiovascular function, oxygen saturation, blood flow, core temperature, respiratory rate, tidal volume, minute ventilation, sounds of the upper airway, heart, and lungs, body position, and activity level. The implanted CMS can produce timely alerts related to clinically significant hypertension, hypotension, BP variability, myocardial ischemia, myocardial infarction, worsening heart failure, cardiac arrhythmias, and worsening pulmonary function due to infection, edema, and pulmonary embolism.

Referring now with more particularity to methods of using the above-described systems, a method of associating a sensor with a blood vessel, a method of continuously measuring the blood pressure waveform, and a method of surgically implanting a sensor within a human body and around the outside wall of a blood vessel are described.

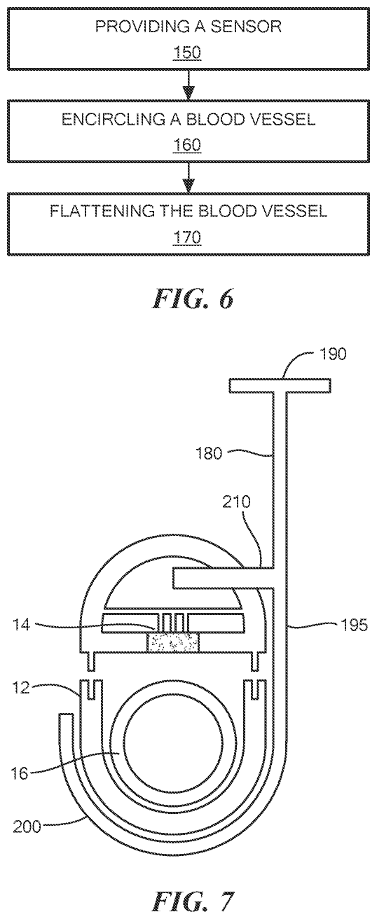

FIG. 6 illustrates steps for a method of associating a sensor with a blood vessel. In a first step 150, a sensor system, (hereinafter “sensor) is provided, such as the combination of the sensor 14 and sensor module retaining device 12, described above with respect to FIGS. 1-5. In a step 160, the sensor is positioned to encircle at least a portion of the blood vessel 16, as shown in FIG. 5. The act of joining two separate portions (the sensor 14 and the sensor module retaining device 12) allows for both easy capture of the blood vessel and placement of the sensor with the pressure/force sensor's transducer in the optimal alignment. Because of the structure of the illustrated components, the diameter of the passage through the sensor can be precisely reduced to cause a portion of the blood vessel to be flattened a specific amount in a step 170 and as shown in FIG. 5.

A surgical implantation tool 180, shown in FIG. 7, can be provided to position the first portion and the second portion of the sensor (the sensor 14 and the sensor module retaining device 12) on opposite sides of the blood vessel 16. Urging the first portion toward the second portion causes them to be joined, thereby completely encircling the blood vessel, and flattening a portion of the blood vessel as the inferior surface of the sensor 14 presses against the outside wall of the blood vessel. The inner diameter of the sensor module retaining device can be increased or decreased as desired by altering the shape of one or both of first or the second portion to accommodate blood vessels of different shape or diameter, and to create a passage size to cause specific applanation of the blood vessel. In an exemplary method, joining the sensor portions causes applanation of the blood vessel in the range of 10% to 25%. Exemplary diameters of the sensor module retaining device range from 2.1 mm to 2.9 mm for implantation the sensor module around the outside of a peripheral artery of an adult human approximately at the level of the aortic valve.

Further with regard to sensor placement, a method of surgically implanting a sensor within a human body includes locating a peripheral artery at the approximate level of an aortic valve, measuring an outer diameter of the peripheral artery, providing the support structure 12 having a tissue ingrowth surface for the peripheral artery, positioning a portion of the peripheral artery within the support structure 12, positioning a sensor over the portion of the peripheral artery with sufficient force to deform the peripheral artery and to join the sensor to the support structure 12, and joining the support structure 12 to the sensor to maintain the deformation of the peripheral artery. Following implantation, the vessel wall tissue (smooth muscle and connective) will remodel over days to weeks to take on the exact shape of the inside passage of the sensor module.

FIG. 7 illustrates an exemplary implantation tool 180 that is useful for positing the sensor and securing it to a selected blood vessel. The tool 180 is shown having a “J” shape, with a handle 190, a shaft 195, a curved, distal capture element 200, and a proximal capture element 210. The size and curvature of the distal capture element 200 can be modified to accommodate larger or smaller sensor modules and sensor module retaining devices as a function of specific blood vessel size. In use, the curved, distal capture element 200 conformally surrounds the sensor module retaining device 12 and the proximal capture element 210 engages the sensor module 14. As the tool 180 is manipulated, such as by twisting the handle 190 to cause the proximal and distal capture elements to be drawn together, the act of doing so causes the sensor module retaining device 12 to be urged towards the sensor module 14 until the two are in contact and secured together, thereby causing a specific degree of applanation of the blood vessel.

The remodeled (flattened) blood vessel wall tissue will not have excessive stress and strains at the corners typical of an acutely flattened artery. The remodeled tissue will facilitate the transfer of intravascular pressure to the pressure sensor's diaphragm without loss of energy or distortion of the pressure waveform.

While the sensor module retaining device 12 can be mechanically or magnetically secured to the sensor 14, the security of the attachment of the elements further comprises promotion of tissue ingrowth into at least a portion of the sensor. The tissue can form a fibrous capsule around the sensor to enhance coupling of the sensor to the blood vessel wall (see photo below of explanted artery wall tissue that surrounds a tonometer sensor head). Fibrous tissue does not develop between the outer surface of the blood vessel wall and the inferior surface of the sensor module.

Once the sensor has been placed, it can be used to continuously measure and accurately measure the arterial blood pressure waveform. Accordingly, and with reference to FIGS. 1-6, a method of measuring blood pressure, is provided including providing a pressure/force sensor defining a passage therethrough in step 150, encircling an artery with at least a portion of the sensor in step 160, and reducing the diameter of the passage through the sensor until a portion of the artery is flattened in step 170. The extravascular pressure/force sensor measures the intravascular arterial blood pressure waveform and outputs a signal to an electronic module that calculates systolic, mean, and diastolic blood pressure based upon the sensed movement. The detailed arterial BP waveform can also be analyzed to determine myocardial contractility, heart rate, stroke volume, arterial blood flow, the forward pressure wave, the amplitude and timing of the reflected pressure wave, systolic/diastolic timing intervals, cardiac valve function, systemic vascular resistance, and hemodynamic significance of a cardiac arrhythmia. Clinicians will use the arterial BP waveform data to produce a more timely and accurate diagnosis, and determine the type of medication, dose of medication, and time of medication delivery to optimize short-term and long-term blood pressure control, leading to a lower risk for myocardial infarction, ischemic stroke, hemorrhagic stroke, heart failure, kidney failure, peripheral vascular disease, dementia, and sudden death.

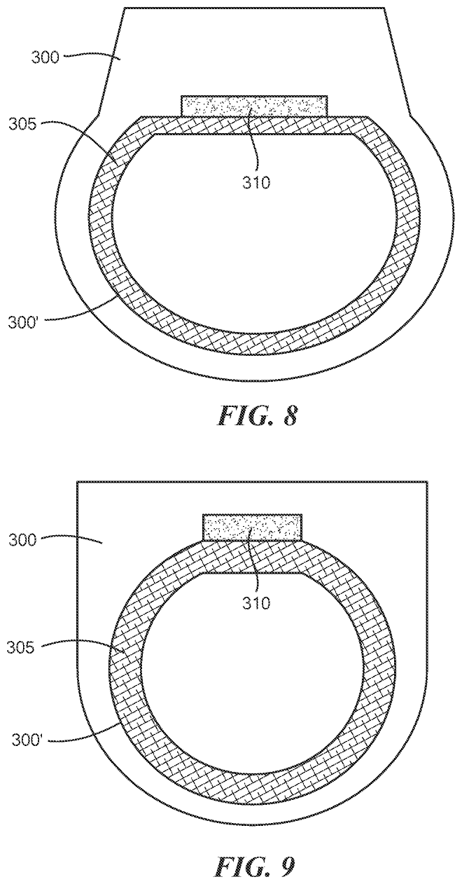

Additional details, features and alternate embodiments of the sensor are shown in FIGS. 8-12. Referring now to FIG. 8, an applanation tonometer sensor head 300 and sensor module retaining device 300′ surrounds the outside wall of a peripheral artery with 15% to 20% applanation, and with the pressure/force transducer or the pressure/force sensor's diaphragm 310 located within the area of applanation.

FIG. 9 illustrates a sensor module 300 attached to a sensor module retaining device 300′ that surrounds the outside wall of a small peripheral artery 305. The shape and diameter of the sensor module 300 and sensor module retaining device 300′ are optimized to align the pressure/force sensor's diaphragm (1 mm width×6 mm length) within a small area of applanation or flattening (<10%). Texturing the inside surface of the sensor module 300 facilitates adhesion of artery wall tissue. The open stent-like structure of the sensor module retaining 310′ device facilitates ingrowth of artery wall tissue and the ingrowth of adjacent vascular tissue. Robust attachment of the artery wall tissue to the sensor module 300 and the sensor module retaining device 300′ prevents localized vasoconstriction, and the loss of mechanical coupling of the diaphragm to the artery wall. This configuration minimizes the effects of applanation on the shape and cross-sectional area of the blood vessel lumen.

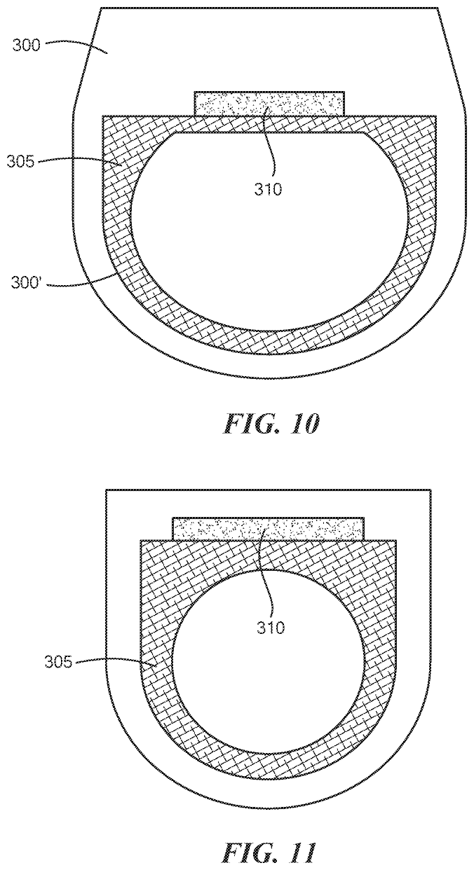

FIG. 10 illustrates a sensor module 300 attached to a sensor module retaining device 300′ that surrounds the outside wall of a small peripheral artery 305. The shape and diameter of the sensor module 300 and sensor module retaining device 300′ are optimized to align the pressure/force sensor's diaphragm (1 mm width×6 mm length) within a larger area of applanation or flattening (15% to 20%). Texturing the inside surface of the sensor module 300 facilitates adhesion of artery wall tissue. The open stent-like structure of the sensor module retaining device 300′ facilitates ingrowth of artery wall tissue and the ingrowth of adjacent vascular tissue. Robust attachment of the artery wall tissue to the sensor module and retaining device prevents localized vasoconstriction, and the loss of mechanical coupling of the diaphragm to the artery wall. Thus, the effects of applanation are minimized on the shape and cross-sectional area of the blood vessel lumen.

FIG. 11 illustrates a sensor module 300 attached to a sensor module retaining device 300′ that surrounds the outside wall of a small peripheral artery 305. Following implantation, the artery wall tissue will remodel to take on the exact shape of the sensor's inner lumen. The artery wall does not undergo applanation and the artery lumen remains round. Artery wall tissue will grow into the corners of the sensor lumen and matures several weeks after surgical implantation. This configuration allows the pressure/force sensor's diaphragm 310 to have a greater surface area (for example a 2 mm width×6 mm length). Texturing the inside surface of the sensor module facilitates the adhesion of artery wall tissue. The open stent-like structure of the sensor module retaining device facilitates ingrowth of artery wall tissue and the ingrowth of adjacent vascular tissue. Robust attachment of the artery wall tissue to the sensor module and retaining device prevents localized vasoconstriction, and the loss of mechanical coupling of the diaphragm to the artery wall. The goal is to minimize the effects of applanation on the shape and cross-sectional area of the blood vessel lumen.

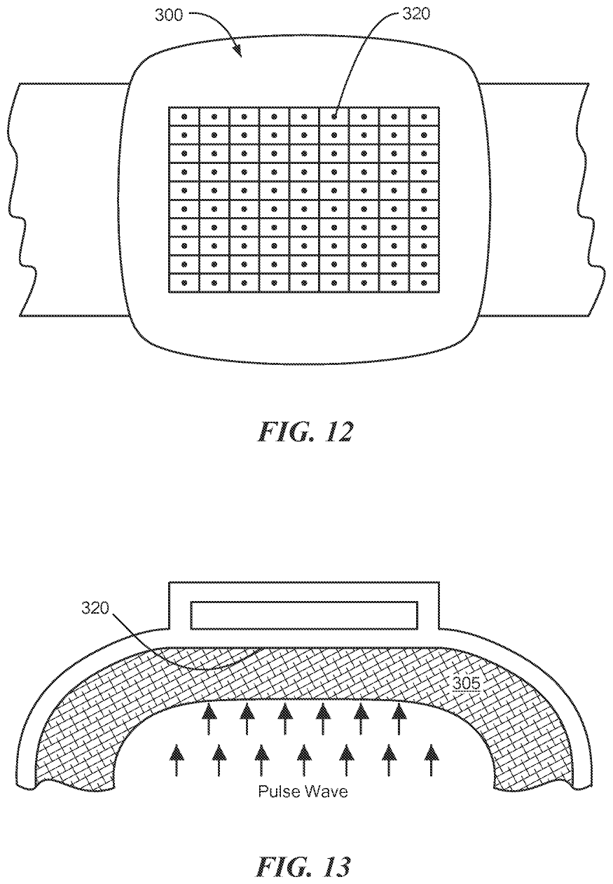

FIG. 12 shows a configuration wherein the inner surface of the sensor module 300 can be made from a single piezoelectric force transducer or an array 320 of force transducers (for example 4, 16, 36, 72, 144 force transducers on a 1 mm×6 mm computer chip) coupled to the outside wall of a peripheral artery within the region of applanation. Each piezoelectric transducer can produce and output signal when pressure/force is applied to their surface as shown in FIG. 13. Multiple output signals can be signal processed and combined to produce a large amplitude and accurate measurement of the arterial BP waveform. Individual output signals from transducers at the edges of applanation with a low signal to noise ratio or a variable amplitude may be ignored.

FIG. 13 shows the sensor module's array of force transducers coupled to the outer blood vessel wall within the area of applanation.

As used herein, relational terms, such as “first” and “second,” “top” and “bottom,” and the like, may be used solely to distinguish one entity or element from another entity or element without necessarily requiring or implying any physical or logical relationship or order between such entities or elements. The terminology used herein is for the purpose of describing particular embodiments only and is not intended to be limiting of the concepts described herein. As used herein, the singular forms “a”, “an” and “the” are intended to include the plural forms as well, unless the context clearly indicates otherwise. It will be further understood that the terms “comprises,” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

In embodiments described herein, the joining term, “in communication with” and the like, may be used to indicate electrical or data communication, which may be accomplished by physical contact, induction, electromagnetic radiation, radio signaling, infrared signaling or optical signaling, for example. One having ordinary skill in the art will appreciate that multiple components may interoperate and modifications and variations are possible of achieving the electrical and data communication.

It will be appreciated by persons skilled in the art that the present embodiments are not limited to what has been particularly shown and described herein above. In addition, unless mention was made above to the contrary, it should be noted that all of the accompanying drawings are not to scale. A variety of modifications and variations are possible in light of the above teachings.

What is claimed is:

1. A method of associating a sensor with a blood vessel, comprising:
providing a sensor defining a passage therethrough;
at least partially encircling the outside wall of the blood vessel with at least a portion of the sensor; and
compressing the passage through the sensor until a portion of the blood vessel is flattened, and
mechanically coupling a pressure/force transducer to the outside wall of the blood vessel in an area of vessel wall flattening.

2. The method of claim 1, wherein the sensor comprises a first portion and a second portion, each defining a portion of the passage through the sensor, for cooperatively encircling the blood vessel, and
providing a surgical implantation tool that positions the first portion and the second portion of the sensor on opposite sides of the blood vessel in a selected x-y-z alignment, and
positioning the sensor within the area of vessel wall flattening.

3. The method of claim 2, further comprising urging the first portion toward the second portion to join the first portion and the second portion, thereby encircling the blood vessel, and flattening a portion of the blood vessel.

4. The method of claim 3, wherein flattening a portion of the blood vessel causes at least 10% applanation of the portion of the blood vessel.

5. The method of claim 3, wherein flattening a portion of the blood vessel causes at least 15% application of the portion of the blood vessel.

6. The method of claim 3, wherein flattening a portion of the blood vessel causes at least 25% application of the portion of the blood vessel.

7. The method of claim 1, further comprising promotion of tissue ingrowth into at least a portion of the sensor to enhance mechanical coupling of the sensor to the blood vessel in the area of flattening.

8. The method of claim 1, wherein the passage therethrough of the sensor encircles the blood vessel with at least a portion of the sensor and reduces the diameter of the passage through of the sensor until a portion of the blood vessel is flattened;
wherein the pressure/force transducer measures pressure within the blood vessel and outputs a signal to an electronic module that analyzes and displays the blood vessel blood pressure waveform and calculates blood pressure based upon a sensed movement.

9. A method of surgically implanting a sensor within a human body, comprising:
locating a peripheral artery at the approximate level of an aortic valve;
measuring an outer diameter of the peripheral artery with ultrasound;
providing a support structure having a tissue ingrowth surface for the peripheral artery;
positioning a portion of the peripheral artery within the support structure;
positioning a sensor module over the portion of the peripheral artery with sufficient force to deform the peripheral artery and to join the sensor module to the support structure; and
joining the support structure to the sensor module to maintain the deformation of the peripheral artery.

10. The method of claim 9, further comprising:
promoting tissue ingrowth of the portion of the peripheral artery sufficient to couple the portion of the peripheral artery to one of the support structure and the sensor.

11. The method of claim 9, wherein the force to deform the portion of the peripheral artery is sufficient to cause at least 10% applanation of the portion of the peripheral artery.

12. The method of claim 9, wherein the force to deform the portion of the peripheral artery is sufficient to cause at least 15% applanation of the portion of the peripheral artery.

13. The method of claim 9, wherein the force to deform the portion of the peripheral artery is sufficient to cause at up to 25% applanation of the portion of the peripheral artery.

14. A method of surgically implanting a sensor within a human body, comprising:
locating a peripheral artery at the approximate level of an aortic valve;
measuring an outer diameter of the peripheral artery with ultrasound;
choosing an optimum size of a sensor module and a support structure;
providing the support structure having a tissue ingrowth surface for the peripheral artery;
positioning a portion of the peripheral artery within the support structure;
positioning the sensor module over the portion of the peripheral artery with sufficient force to deform the peripheral artery and to join the sensor module to the support structure; and
joining the support structure to the sensor module to maintain the deformation of the peripheral artery; and
mechanically securing an array of force transducers to the outer surface of the blood vessel wall.

15. The method of claim 14, wherein when the sensor module is positioned over the portion of the peripheral artery with sufficient force to deform the peripheral artery and the sensor module is joined to the support structure, the sensor module and the support structure cooperatively encircle the blood vessel.
